(19) United States
(12) Patent Application Publication
(10) Pub. No.: US 2007/0142395 A1
(43) Pub. Date: Jun. 21, 2007

(54) Treatment of sexual dysfunction with 1-[3-[4-(3-chlorophenyl)-1-piperazinyl]propyl]-5-methoxy-3,4-dihydro-2(1H)-quinolone or salt thereof

(75) Inventors: Steven M. Leventer, Langhorne, PA (US); Robert F. Kucharik, Glenmoore, PA (US); Jeff Calcagno, Hopewell, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Vela Pharmaceuticals, Inc., Ewing, NJ (US); Otsuka Pharmaceutical Co., Ltd., Tokyo (JP)

(21) Appl. No.: 11/305,277

(22) Filed: Dec. 16, 2005

(51) Int. Cl. A61K 31/496

(52) U.S. Cl. 514/253.07

(57) ABSTRACT

Therapeutic methods are provided for treating sexual dysfunction by administration of 1-[3-[4-(3-chlorophenyl)-1-piperazinyl]propyl]-5-methoxy-3,4-dihydro-2(1H)-quinolone, or a prodrug or salt thereof.

### FIELD OF THE INVENTION

The present invention relates to methods of treating sexual dysfunction, in particular methods of treatment for sexual dysfunction having at least one of reduced sexual interest, reduced sexual desire, reduced sexual pleasure and reduced sexual arousal and excitement.

### BACKGROUND OF THE INVENTION

Certain carbostyryil derivatives are described in U.S. Pat. Nos. 5,556,857 and 5,656,633. Among the disclosed compounds are 1-[3-[4-(3-chlorophenyl)-1-piperazinyl]propyl]-5-methoxy-3,4-dihydro-2(1H)-quinolone and salts therof. The monomethanesulfonate, known as “OPC-14523”, has been used as an antidepressant. The aforesaid carbostyryil derivatives are not known for the treatment of sexual dysfunction.

Sexual Dysfunction

The set of physiological and emotional changes that lead to and follow orgasm are referred to as “sexual response”. Clinical study of sexual response and sexual dysfunction is relatively recent in the medical field. It is generally agreed to have been birthed by the publication of the work of Masters and Johnson: Human Sexual Response (Masters and Johnson, Lippincott Williams & Wilkins, 1966) and Human Sexual Inadequacy (Masters, Little Brown & June, 1970). The sexual response cycle can be divided into 4 phases: desire, excitement, orgasm and resolution. The desire (or libido) phase involves fantasies about sexual activity and the desire to have sexual activity or gratification. The excitement (or arousal) phase involves a subjective sense of sexual pleasure and accompanying physiological changes, including vasoconstriction and myotonia. The orgasm phase consists of a peaking of sexual pleasure, with release of sexual tension and rhythmic contraction of the perineal muscles and reproductive organs. In males, there is a feeling of ejaculatory inevitability, followed by ejaculation. In females, there are contractions of the wall of the outer third of the vagina. The resolution phase consists of a sense of muscle relaxation and general well-being.

Sexual dysfunction is a condition characterized by a disturbance in any process involved in the sexual response cycle that reduces the person's sexual enjoyment. Sexual dysfunction disorders are a group of disorders generally characterized by sexual dysfunction which causes marked personal stress. Diagnostic criteria have been established for various sexual dysfunction disorders (Diagnostic and Statistical Manual of Mental Disorders, Fourth Edition, Text Revision. American Psychiatric Association, Washington, D.C., 2000, pp. 535-566; referred to herein as “DSM-IV-TR”). The four main categories of the disorders are: Sexual Desire Disorders; Sexual Arousal Disorders; Orgasmic Disorders; and Sexual Pain Disorders. Other categories are Sexual Dysfunction Due to a General Medical Condition; Substance-Induced Sexual Dysfunction; and Sexual Dysfunction Not Otherwise Specified (NOS). Disturbances can occur at one or more of the phases of the sexual response cycle. Thus, a patient may be diagnosed with more than one sexual disorder.

The prevalence of sexual dysfunction is high. Forty to forty-five percent of adult women and 20% to 30% of adult men have at least one manifest sexual dysfunction (Lewis et al., J. Sex. Med. 1(1):35-39 (2004)). Hypoactive Sexual Desire Disorder (“HSDD”), characterized by reduced sexual interest and sexual desire, is one of the most common sexual dysfunction disorders in both women and men (Rosen, Curr. Psychiatry Rep. 2(3):189-95 (2000)). It is estimated that about one-third of women have HSDD (Diagnostic and Statistical Manual of Mental Disorders, Fourth Edition, Text Revision. American Psychiatric Association, Washington, D.C., 2000, pp. 535-566). Some experts in the field believe that the prevalence of HSDD in men is underestimated because it is misinterpreted as erectile dysfunction (Meuleman et al., BJU International 95:291-2936 (2005)).

Sexual dysfunction is a common side effect of selective serotonin reuptake inhibitors (SSRIs) and serotonin-norepinephrine reuptake inhibitors (SNRIs). The overall rate of sexual dysfunction as a side effect of SSRI/SNRI antidepressant therapy is about 59% (Montejo et al., J. Clin. Psychiatry 62 Suppl 3:10-21 (2001)). Montejo et al. also reported the incidence of sexual dysfunction for specific SSRIs/SNRIs including fluoxetine (57.7%), sertraline (62.9%), fluvoxamine (62.3%), paroxetine, (70.7%), citalopram (72.7%) and venlafaxine (67.3%).

There are few pharmacological treatments for sexual dysfunctions. Some patients with male erectile disorder (“ED”) can be treated successfully by PDE-5 inhibitors, e.g., sildenafil and tadalafil. In some instances, HSDD due to hormonal deficiencies may be treated successfully by hormonal regimens. In addition, there is also some evidence that buproprion may be effective in treating HSDD (Segraves et al., J Clin. Psychopharmacol. 24(3):339-342 (2004)). In general, however, there are very few sexual dysfunction therapeutics. Given the high prevalence of sexual dysfunction overall, there is a clear need in the art for a treatment for sexual dysfunction.

### SUMMARY OF THE INVENTION

According to the present invention, therapeutic methods are provided for sexual dysfunction. The method comprises administering to the individual an effective amount of 1-[3-[4-(3-chlorophenyl)-1-piperazinyl]propyl]-5-methoxy-3,4-dihydro-2(1H)-quinolone, a prodrug thereof, or a pharmaceutically acceptable salt thereof. The preferred salt is the monomethanesulfonate. The monomethoansulfonate of 1-[3-[4-(3-chlorophenyl)-1-piperazinyl]propyl]-5-methoxy-3,4-dihydro-2(1H)-quinolone is also known as “OPC-14523”.

According to some embodiments of the sexual dysfunction treatment method of the invention, a measure of the sexual dysfunction includes reduced sexual interest. In other embodiments of the sexual dysfunction treatment method, a measure of the sexual dysfunction includes reduced sexual desire. In yet other embodiments of the sexual dysfunction treatment method, a measure of the sexual dysfunction includes reduced sexual pleasure. In yet other embodiments of the sexual dysfunction treatment method, a measure of the sexual dysfunction includes reduced sexual arousal and excitement. In yet other embodiments of the sexual dysfunction treatment method, the sexual dysfunction is HSDD. In yet other embodiments of the sexual dysfunction treatment method, the sexual dysfunction is ED.

According to another aspect of the invention, 1-[3-[4-(3-chlorophenyl)-1-piperazinyl]propyl]-5-methoxy-3,4-dihydro-2(1H)-quinolone, a prodrug thereof, or a pharmaceutically acceptable salt thereof, is used in the preparation of a medicament for treating sexual dysfunction.

### DEFINITIONS

As used in the specification and the appended claims, the singular forms “a,” “an” and “the” include plural referents unless the context clearly dictates otherwise.

The terms “effective amount” or “pharmaceutically effective amount” refer to a nontoxic but sufficient amount of an agent to provide the desired biological result. That result can be reduction and/or alleviation of the signs, symptoms, or causes of a disease, or any other desired alteration of a biological system. An appropriate “effective” amount in any individual case may be determined by one of ordinary skill in the art using routine experimentation.

As used herein, the terms “treat” or “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

As used herein, “sexual dysfunction” refers to a condition having one or more symptoms of difficulty during any one or more stages (desire, arousal, orgasm and resolution) of the sexual act that prevents an individual from enjoying sexual activity. Non-limiting examples of symptoms of sexual dysfunction include: reduced sexual desire, reduced sexual interest, reduced sexual pleasure, reduced sexual arousal and excitement, aversion to and avoidance of genital sexual contact, inability to attain or maintain arousal, and persistent or recurrent delay of, or absence of orgasm. Sexual dysfunction may be lifelong (no effective performance ever) or acquired (after a period of normal function); generalized or limited to certain situations or certain partners; and total or partial.

As used herein, “sexual desire” refers to the frequency of engaging in sexual activity and/or the frequency of desiring to engage in sexual activity as perceived by the individual.

As used herein, “sexual interest” refers to sexual desire as expressed in cognitive activity, including the frequency of sexual thoughts, the extent of enjoyment of movies, books, music, etc having sexual content and/or the extent of enjoyment or pleasure of thinking and fantasizing about sex as perceived by the individual.

As used herein, “sexual pleasure” refers to the extent of enjoyment of one's current sexual activity as a whole.

As used herein, “sexual arousal and excitement” refers to the frequency of becoming sexually aroused, how readily sexual arousal occurs and/or if arousal is maintained, as perceived by the individual.

As used herein, “individual” (as in the subject of the treatment) means both mammals and non-mammals. Mammals include, for example, humans; non-human primates, e.g., apes and monkeys; cattle; horses; sheep; and goats. Non-mammals include, for example, fish and birds.

### DESCRIPTION OF THE FIGURES

FIG. 1 depicts the difference in the Changes in Sexual Function Questionaire (CSFQ) scores (difference from placebo) from baseline to week 8 for OPC-14523, buproprion XL and escitalopram. The data for bupropion XL and escitalopram were obtained from Clayton et al., 45^{th} Annual NCDEU Meeting, Session 1-21, Boca Raton, Fla., June 6-9, 2005. For the Clayton et al. data, N=273 (placebo), 276 (bupropion XL, 300-400 mg) and 281 (escitalopram, 10-20 mg).

### DETAILED DESCRIPTION OF THE INVENTION

According to the present invention, 1-[3-[4-(3-chlorophenyl)-1-piperazinyl]propyl]-5-methoxy-3,4-dihydro-2(1H)-quinolone, a prodrug thereof, or a pharmaceutically acceptable salt thereof, is administered to treat sexual dysfunction.

1-[3-[4-(3-Chlorophenyl)-1-piperazinyl]propyl]-5-methoxy-3,4-dihydro-2(1H)-quinolone has the formula 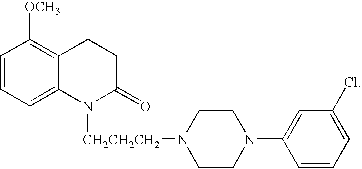
The compound and its pharmaceutically acceptable salts may be prepared according to the synthetic methods described in U.S. Pat. Nos. 5,556,857 and 5,656,633 and Oshiro et al., J. Med. Chem. 43:177-189 (2000), the entire disclosures of which are incorporated herein by reference. The monomethanesulfonate is compound 34c of Oshiro et al.

The compound 1-[3-[4-(3-chlorophenyl)-1-piperazinyl]propyl]-5-methoxy-3,4-dihydro-2(1H)-quinolone, prodrug thereof, or pharmaceutically acceptable salt thereof, may be administered to any subject for treating sexual dysfunction.

The compounds used in the practice of methods of the present invention may take the form of pharmaceutically acceptable salts. The term “salts” embraces salts commonly used to form alkali metal salts and to form addition salts of free acids or free bases. The term “pharmaceutically acceptable salt” refers to salts that possess toxicity profiles within a range so as to have utility in pharmaceutical applications. Suitable pharmaceutically acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, beta-hydroxybutyric, galactaric, and galacturonic acid.

The compound may also take the form of a prodrug of 1-[3-[4-(3-chlorophenyl)-1-piperazinyl]propyl]-5-methoxy-3,4-dihydro-2(1H)-quinolone. Prodrugs according to this invention are inactive derivatives of 1-[3-[4-(3-chlorophenyl)-1-piperazinyl]propyl]-5-methoxy-3,4-dihydro-2(1H)-quinolone that are metabolized in vivo into the active agent in the body. Prodrugs useful according to this invention are those that have therapeutic value substantially the same or better than 1-[3-[4-(3-chlorophenyl)-1-piperazinyl]propyl]-5-methoxy-3,4-dihydro-2(1H)-quinolone in treating sexual dysfunction. For example, a prodrug useful according to this invention can improve the penetration of the drug across biological membranes, leading to improved drug absorption; prolong duration of the action of the drug, e.g., slow release of the parent drug from the prodrug and/or decrease first-pass metabolism of the drug; target the drug action; improve aqueous solubility and stability of the drug (e.g., intravenous preparations); improve topical drug delivery (e.g., dermal drug delivery); improve the chemical and/or enzymatic stability of drugs; or decrease side effects due to the drug. Methods for making prodrugs are know in the art (e.g., Balant, Eur. J. Drug Metab. Pharmacokinet. 15:143-153 1990; and Bundgaard, Drugs of the Future 16:443-458, 1991; incorporated herein by reference).

Sexuality comprises both physiologic and psychogenic input. Physiologic input as used herein refers to functions of the body, including vascular function, neurological function and hormonal function. Psychogenic input as used herein refers to psychological input, that is, input originating in or from the mind. Sexual dysfunction consequently can arise from a problem in one or both sources of input. While not wishing to be bound by theory, it is believed that the compounds of the invention are effective in treating sexual dysfunction by altering or correcting psychogenic input.

Assessment of sexual function, including assessment of specific measures of sexual function such as sexual desire, sexual interest, sexual arousal and excitement, and/or sexual pleasure, may be carried out using any reliable method known to the skilled artisan. Examples of such methods include evaluation by a clinician trained in sexual health and validated, self-report questionnaires about sexual function. Examples of such questionnaires include, but are not limited to, Changes in Sexual Function Questionaire (“CSFQ”; Clayton et al., Psychopharmacol Bull. 33(4):731-45 (1997) and Clayton et al., Psychopharmacol. Bull. 33(4):747-53 (1997)); the Derogatis Interview for Sexual Functioning—Self-Report (“DISF-SR”; Derogatis, J Sex Marital Ther. 23:291-304 (1997)); the Female Sexual Function Index (“FSFI”; Rosen et al., J Sex Marital Ther. 26:191-208 (2000)); the Golombok-Rust Inventory of Sexual Satisfaction (“GRISS”; Rust et al., Arch. Sex Behav. 15:157-165 (1986)); the Sexual Function Questionnaire (“SFQ”; Quirk et al., J Womens Health Gend Based Med. 11:277-289 (2002)); and the Arizona Sexual Experience Scale (“ASEX”; McGahuey et al., J Sex Marital Ther. 26:25-40 (2000)), the entire disclosures of which are incorporated herein by reference.

For assessment using a questionnaire, a measure of sexual function is “reduced” when the score in the appropriate domain, subscale or subtest is indicative of sexual dysfunction, as established for that questionnaire. For instance, a female's sexual interest is considered reduced, when assessed using the CSFQ, if the subscale for sexual interest score is less than or equal to 9 (see Table 4). For a clinician's assessment, “reduced” may be in comparison to a previous point in time for the patient and/or in comparison to a patient's peers with respect to age, gender, sexual experience, and health, or may also be determined via a validated questionnaire administered by the clinician.

The compounds of the invention are useful in treating sexual dysfunction. As the CSFQ Total Score data herein indicate, treatment with OPC-14523 improves the overall global sexual functioning of men and women. Thus, the compounds of the invention are useful in treating sexual dysfunction disorders.

The compounds of the invention are particularly useful in treating sexual dysfunction that includes reduced sexual interest and/or reduced sexual desire as symptoms. A non-limiting example of sexual dysfunction related to reduced sexual interest and/or desire is HSDD. HSDD is classified as a Sexual Desire Disorder in the DSM-IV-TR and is one of the most common sexual dysfunctions in both men and women. According to the DSM-IV-TR, the essential feature of HSDD is a persistent or recurring deficiency or absence of sexual fantasies and persistence or recurring absence of desire for sexual activity. A person with HSDD typically does not initiate sexual activity or seek sexual activity. The reduction or absence of sexual desire, sexual interest, and/or sexual fantasies causes extreme personal distress. Treatment with the compounds of the invention leads to a significant improvement in both sexual desire and sexual interest. Notably, both men and women undergoing treatment with OPC-14523 had improved sexual interest (p=0.07 and p=0.08, respectively). Therefore, the compounds of the invention are particularly effective in treating HSDD in both men and women.

The compounds of the invention are also particularly useful in treating sexual dysfunction that includes reduced sexual pleasure and/or reduced sexual arousal and excitement as a symptom. A non-limiting example of sexual dysfunction with such symptoms is ED. The essential feature of ED, a Sexual Arousal Disorder, is a persistent or recurrent inability to attain an adequate erection or maintain an adequate erection until the completion of sexual activity. It is frequently associated with a decreased subjective sense of sexual pleasure and excitement. It may also be associated with HSDD. Men undergoing treatment with OPC-14523 experienced a strong trend of improved sexual pleasure and sexual arousal and excitement. Therefore, the compounds of the invention are effective in treating ED associated with reduced sexual pleasure and/or reduced arousal and excitement.

Sexual dysfunction may also be substance-induced, such as an SSRI or SNRI, and is also frequently present in patients who are diagnosed as depressed, e.g. Major Depressive Disorder (“MDD”) or have an anxiety disorder, e.g. Generalized Anxiety Disorder (“GAD”). When such sexual dysfunction includes a symptom of reduced sexual desire, reduced sexual interest, reduced sexual pleasure, and reduced sexual arousal and excitement, the compounds of the invention are useful in treating the sexual dysfunction.

For treating sexual dysfunction, the specific dose of compound according to the invention to obtain therapeutic benefit will, of course, be determined by the particular circumstances of the individual patient including the size, weight, age and sex of the patient, the nature and extent of the condition being treated, and recommendations of the treating physician. Also determinative will be the stage of the disease and the route of administration. Thus, the effective amount for a given situation can be determined by routine experimentation. Generally a therapeutic amount will be in the range of about 0.01 mg/kg to about 40 mg/kg body weight, more preferably about 0.1 mg/kg to about 10 mg/kg, in at least one dose. In larger mammals, the indicated dosage can be from about 1 mg to 1500 mg, one or more times per day, more preferably in the range of about 1 mg to 100 mg, and more preferably still, in the range of about 2.5 mg to 80 mg. Higher or lower doses are also contemplated. The subject may be administered as many doses as is required to reduce and/or to alleviate the signs, symptoms, or causes of the disorder in question. The frequency of dosing may, for instance, be once, twice or thrice daily. Treatment duration may, for instance, be 2, 4 or 8 weeks, 6 to 12 months, or more. Both episodic and chronic treatment may be undertaken as necessary. A non-limiting example of a treatment regimen is twice daily oral doses of 5 mg per dose, administered for 6 to 12 months.

The methods of the present invention may comprise administering the compound in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent, or excipient that is compatible with the other ingredients of the formulation and not deleterious to the recipient.

The compound may be administered for therapeutic effect by any route, for example enteral (oral, rectal, intranasal, etc.) and parenteral administration. Parenteral administration includes, for example, intravenous, intramuscular, intraarterial, intraperitoneal, intravaginal, intravesical (for example, into the bladder), intradermal, topical, and subcutaneous administration. Also contemplated within the scope of the invention is the instillation of drug in the body of the patient in a controlled formulation, with systemic or local release (such as, for example, in the gastrointestinal tract) of the drug to occur at a later time.

The active agent is preferably administered with a pharmaceutically acceptable carrier selected on the basis of the chosen route of administration and standard pharmaceutical practice. The active agent may be formulated into dosage forms according to standard practices in the field of pharmaceutical preparations. (See Gennaro, editor, Remington's Pharmaceutical Sciences, 18th ed., Easton, Pa., Mack Publishing Co., 1990. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water-soluble salt of the active agent. Stabilizing agents, antioxidizing agents and preservatives may also be added. Suitable antioxidizing agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents, or lubricating agents. According to one tablet embodiment, the active agent may be combined with carboxymethylcellulose calcium, magnesium stearate, mannitol and starch, and then formed into tablets by conventional tableting methods.

The compositions of the present invention can also be formulated so as to provide slow or controlled release of the active ingredient therein. In general, a controlled-release preparation is a composition capable of releasing the active ingredient at the rate required to maintain constant pharmacological activity for a desirable period of time. Such dosage forms can provide a supply of a drug to the body during a predetermined period of time and thus maintain drug levels in the therapeutic range for longer periods of time than other non-controlled formulations.

The controlled release of the active ingredient may be stimulated by various inducers, for example, pH, temperature, enzymes, water, or other physiological conditions or compounds. Various mechanisms of drug release exist. For example, in one Embodiment, the controlled-release component can swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient (for example, the compound or a pharmaceutically acceptable salt thereof) in the pharmaceutical composition. In another embodiment, the controlled-release component is biodegradable, induced by exposure to the aqueous environment, pH, temperature, or enzymes in the body. In another embodiment, sol-gels can be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The compound is administered according to the present invention to patients suffering from symptoms of sexual dysfunction. Such conditions include, for example, HSDD and ED.

The practice of the invention is illustrated by the following non-limiting examples.

### EXAMPLES

### Example 1

### Preparation of Immediate Release Tablet

Tablets were prepared containing 5 mg active ingredient (OPC-14523). The tablets contained, in addition to the active ingredient, lactose monohydrate (101.0 mg), corn starch (20.0 mg), microcrystalline cellulose (20.0 mg), low-substituted hydroxypropyl cellulose (20.0 mg), hydroxypropyl cellulose (4.0 mg), hydrogenated castor oil (4.0 mg), hydroxypropyl methyl cellulose (4.8 mg), titanium dioxide (0.6 mg), talc (0.6 mg) and purified water (145.0 mg).

### Example 2

### Effect of OPC-14523 Administration on Sexual Dysfunction in Humans

Materials and Methods

Clinical Study Details:

A double-blind, randomized, placebo-controlled, parallel-group study was performed to evaluate the efficacy, safety and tolerability of OPC-14523 in patients with MDD. The patients selected for participation in the trial had to meet DSM-IV-TR criteria for MDD (296.2x or 296.3x; diagnosis via MINI) and had had symptoms of depression for at least 2 months before the start of the double-blind treatment. Among other drugs prohibited during the study, patients were prohibited from taking drugs used to enhance sexual function, including, but not limited to, sildenafil, tadalafil and vardenafil, or taking psychopharmacologic drugs.

There were 131 patients assigned to receive OPC-14523 at the start of the study. One hundred twenty seven (127) of these patients took at least one dose of OPC-14523. There were 137 patients assigned to receive placebo at the start of the study. One hundred thirty three (133) of these patients took at least one dose of placebo. The baseline demographics for the patients who took at least one dose of OPC-14523 or placebo are summarized in Table 3.

TABLE 3

| | OPC-14523 | Placebo |
|---|---|---|
| Parameter | (N = 127) | (N = 133) |
| Age (years) | 39 | 42 |
| Gender (% female) | 58% | 59% |
| Race (% Caucasian) | 73% | 74% |
| Duration of current episode* | 54 weeks | 53 weeks |
| Hx* of treatment for MDD | 58% | 68% |

*“Current episode” refers to symptoms of depression. “Hx” is history.

Study Schedule:

Each patient was expected to participate for up to 10 weeks. The 10 weeks included: i) a 4- to 10-day screening period, during which potential participants were administered placebo in a single-blind manner; ii) an 8-week double-blind treatment period; and iii) a 4- to 10- day post-treatment visit period. Baseline evaluations, including assessment of sexual function, were conducted on Day −1. The next day, Day 1, the double-blind medication was begun. All treatment was on an outpatient basis.

All patients signed informed consent forms prior to any study-related screening procedures.

Dosage:

The dosages studied were 5 mg BID OPC-14523 (10 mg total daily dose of immediate release tablet of Example 1) and placebo. The drugs were taken after meals in the morning and in the evening, about 12 hours apart.

Sexual Function Data:

Data on sexual function were collected using the Changes in Sexual Function Questionnaire (CSFQ). The CSFQ is a 14-item self-report measure of sexual functioning vis-â-vis experiences of pleasure, desire, arousal, and orgasm, with a 5-point response option for each item. The 14-item validated CSFQ was developed from a questionnaire comprising 35 and 36 items for female and male respondents, respectively (Clayton et al., Psychopharmacol Bull. 33(4):731-45 (1997) and Clayton et al., Psychopharmacol Bull. 33(4):747-53 (1997), each incorporated herein by reference in its entirety). The CSFQ has also been independently validated (Bobes et al., J Sex Marital Ther. 26(2): 119-31(2000)). The 14-item CSFQ includes items 8, 11, 14, 19-24 from the interview version of the CSFQ (“CSFQ-I”); Clayton et al., Psychopharmacol Bull. 33(4):731-45 (1997)). The CSFQ differs for men and women, reflecting some variation in wording of items. Males were given the male clinical version, CSFQ-M-C, and women were given the female clinical version, CSFQ-F-C. CSFQ-F-C includes items 25, 27, 28, 30 and 31 from the questions for women on the CSFG-I. CSFQ-M-C includes items 28-32 from the questions for men on the CSFQ-I, as well as item 25 from the questions for women.

The 14-item CSFQ has subscales for sexual pleasure (1 item), desire/frequency (2 items), desire/interest (3 items), arousal (3 items), and orgasm (3 items). In addition, a 14-item total score may be calculated. Higher scores are indicative of more favorable sexual functioning. Threshold scores for total and sub-scales have been defined. A score below the threshold score indicates dysfunction. The threshold values are shown in Table 4.

TABLE 4

| CSFQ Subscale or | | Threshold Score* | Threshold Score* |
|---|---|---|---|
| Total Score | Item No (s). | for Men | for Women |
| Pleasure | 1 | ≦4 | ≦4 |
| Desire/Frequency | 2 + 3 | ≦8 | ≦6 |
| Desire/Interest | 4 + 5 + 6 | ≦11 | ≦9 |
| Arousal/Excitement | 7 + 8 + 9 | ≦13 | ≦12 |
| Orgasm/Completion | 11 + 12 + 13 | ≦13 | ≦11 |
| Total Score | 1 through 14 | ≦47 | ≦41 |

*Threshold score indicates the score below which a patient is considered to have dysfunction.

Data Analysis:

Results were scored and analyzed according to the CSFQ instruction manual. The p-values resulted from an analysis of covariance (ANCOVA) linear model where the dependent variable is the change from baseline and independent variables are site effect, treatment effect, and baseline. The p-value is from the Type III sums of squares t-test for the hypothesis that the treatment groups are the same versus the alternative they are different. Last observation carry-forward (LOCF) analysis was used as indicated. In the LOCF analysis, for patients who dropped out before completion of the study, the last observations were carried forward to the final time point. Thus, the LOCF analysis treats the carried-forward data as observed data at the last time point.

Data were also analyzed by shift analysis using shift tables. The construction of shift tables is a routine analysis that is often carried out in the analysis of clinical trial data. For the data herein, the analysis was constructed by categorizing the subjects' scores at baseline and then separately at Day 56 according to having sexual dysfunction (SD) or no sexual dysfunction (NSD). These categories were defined by the threshold values specified in the CSFQ (see Table 4). The cross-classification of the baseline categories with the Day 56 categories results in a 2×2 table with the cells NSD/NSD, NSD/SD, SD/NSD, and SD/SD. Two categories represent the number and percent of subjects who stayed in the same category from baseline to Day 56 (NSD/NSD and SD/SD cells). The other two categories represent the number and percentage of subjects who shifted either for better (SD/NSD cell) or worse (NSD/SD cell). In the tables below, “remained low” refers to the SD/SD category. “Remained normal” refers to the NSD/NSD category. “Improved” refers to the SD/NSD category. “Worsened” refers to the NSD/SD category. The p-value was derived from the Chi-square statistic testing the hypothesis that the distribution of the counts and percentages was the same across the 4 categories for the two treatment groups against the alternative hypothesis that the distributions differed between the groups. The generation of the shift tables and the p-values were carried out using SAS® software.

Results t0054] Table 5 shows the baseline mean values for each subscale of the CSFQ, as well as the total score. The data are separated by gender and by drug assignment. Virtually all of the mean scores are indicative of sexual dysfunction in the patients in both treatment groups, prior to the administration of OPC-14523 or placebo.

TABLE 5

| CSFQ Threshold Scores | Baseline- | Baseline- |
|---|---|---|
| (Males/Females) | Males (Mean) | Females (Mean) |
| Pleasure (≦4/4) | | |
| OPC-14523 | 2.2 | 2.0 |
| Placebo | 2.4 | 1.7 |
| Desire/Frequency (≦8/6) | | |
| OPC-14523 | 5.8 | 4.7 |
| Placebo | 5.6 | 4.3 |
| Desire/Interest (≦11/9) | | |
| OPC-14523 | 8.2 | 6.7 |
| Placebo | 8.1 | 6.2 |
| Arousal/Excitement (≦13/12) | | |
| OPC-14523 | 10.5 | 7.9 |
| Placebo | 9.8 | 7.5 |
| Orgasm/Completion (≦13/11) | | |
| OPC-14523 | 11.3 | 8.2 |
| Placebo | 10.1 | 8.6 |
| Total Score (≦47/41) | | |
| OPC-14523 | 47.6 | 37.8 |
| Placebo | 45.5 | 36.4 |

The data in Table 6 show the summary data for all the patients at the three timepoints when the CSFQ data was obtained. These data demonstrate that compared with placebo, at Day 28 and/or Day 56, OPC-14523 significantly improved sexual function overall, as measured by the CSFQ Total Scores. Furthermore, OPC-14523 significantly improved pleasure, sexual desire/frequency, and sexual desire/interest. There was also a trend for OPC-14523 to improve arousal/excitement and orgasm/completion more than placebo.

TABLE 6

| CSFQ All Patients | Base- | | |
|---|---|---|---|
| (Men and Women) | line | Day 28 | Day 56 |
| Pleasure | | | |
| OPC-14523 | 2.06 | 2.41** | 2.51 (p = 0.11) |
| Placebo | 1.97 | 2.05 | 2.23 |
| Desire/Frequency | | | |
| OPC-14523 | 5.13 | 5.39 | 5.57* |
| Placebo | 4.86 | 5.01 | 4.98 |
| Desire/Interest | | | |
| OPC-14523 | 7.30 | 7.66 | 7.94* |
| Placebo | 6.98 | 7.22 | 7.02 |
| Arousal/Excitement | | | |
| OPC-14523 | 8.97 | 9.24 | 9.55 |
| Placebo | 8.48 | 8.69 | 8.79 |
| Orgasm/Completion | | | |
| OPC-14523 | 9.50 | 9.56 | 9.77 |
| Placebo | 9.20 | 9.35 | 9.27 |
| Total Score | | | |
| OPC-14523 | 41.9 | 43.4 | 44.4 (p = 0.05) |
| Placebo | 40.2 | 41.0 | 41.1 |

*p < 0.05 and
**p < 0.01 (change from baseline, OPC-14523 vs. placebo); LOCF

Table 7 shows the summary of data for men only, at the three timepoints when sexual function data were collected. These data indicate that in men, compared with placebo, at Day 28 and/or Day 56, OPC-14523 significantly improved sexual function overall, as well as pleasure, sexual desire/frequency, and sexual desire/interest. There was also a trend for OPC-14523 to improve arousal/excitement more than placebo.

TABLE 7

| CSFQ (Men only) | Baseline | Day 28 | Day 56 |
|---|---|---|---|
| Pleasure | | | |
| OPC-14523 | 2.15 | 2.68*** | 2.60 (p = 0.10) |
| Placebo | 2.36 | 2.19 | 2.44 |
| Desire/Frequency | | | |
| OPC-14523 | 5.77 | 6.26* | 6.38* |
| Placebo | 5.64 | 5.67 | 5.72 |
| Desire/Interest | | | |
| OPC-14523 | 8.21 | 8.94* | 8.91 (p = 0.07) |
| Placebo | 8.11 | 8.13 | 8.10 |
| Arousal/Excitement | | | |
| OPC-14523 | 10.48 | 11.13 (p = 0.07) | 11.17 (p = 0.13) |
| Placebo | 9.82 | 9.81 | 9.98 |
| Orgasm/Completion | | | |
| OPC-14523 | 11.33 | 11.32 | 11.34 |
| Placebo | 10.11 | 10.35 | 10.30 |
| Total Score | | | |
| OPC-14523 | 47.6 | 50.1* | 50.2 (p = 0.12) |
| Placebo | 45.5 | 45.8 | 46.1 |

*p < 0.05,
**p < 0.01 and
***p < 0.001 (change from baseline, OPC-14523 vs. placebo); LOCF

Table 8 shows the summary of data for women only, at the three timepoints when sexual function data were collected. These data indicate, compared with placebo, at Day 28 and/or Day 56, a strong trend by OPC-14523 to improve sexual desire/interest in women. The data also show the overall trend of more improved pleasure, sexual desire/frequency, arousal/excitement, orgasm/completion, and overall sexual function with OPC-14523, compared to placebo.

TABLE 8

| CSFQ (Women only) | Baseline | Day 28 | Day 56 |
|---|---|---|---|
| Pleasure | | | |
| OPC-14523 | 1.99 | 2.22 | 2.46 |
| Placebo | 1.69 | 1.96 | 2.08 |
| Desire/Frequency | | | |
| OPC-14523 | 4.68 | 4.78 | 5.01 |
| Placebo | 4.31 | 4.55 | 4.46 |
| Desire/Interest | | | |
| OPC-14523 | 6.66 | 6.76 | 7.26 (p = 0.08) |
| Placebo | 6.17 | 6.59 | 6.25 |
| Arousal/Excitement | | | |
| OPC-14523 | 7.91 | 7.91 | 8.43 |
| Placebo | 7.52 | 7.91 | 7.96 |
| Orgasm/Completion | | | |
| OPC-14523 | 8.21 | 8.33 | 8.69 |
| Placebo | 8.56 | 8.65 | 8.55 |
| Total Score | | | |
| OPC-14523 | 37.8 | 38.7 | 40.4 |
| Placebo | 36.4 | 37.7 | 37.6 |

*p < 0.05,
**p < 0.01 and
***p < 0.001 (change from baseline, OPC-14523 vs. placebo); LOCF

The sexual function data were also subjected to shift analysis. The results of this analysis for all the patients, for men only and for women only are shown in Tables 9, 10 and 11, respectively. In each case, the shift analysis data support the overall efficacy of OPC-14523 in treating overall sexual dysfunction in men and women. The shift analysis data also support the efficacy of OPC-14523 in improving sexual pleasure, sexual desire, sexual interest, sexual arousal, and orgasm.

In summary, OPC-14523 had an advantage over placebo not only on total CSFQ but on every subscale, including orgasm/completion. The effect of OPC-14523 is especially notable given that OPC-14523 did not demonstrate a statistically significant effect on MDD, the main indication in the clinical trial. Unlike drugs, such as sildenafil, that induce specific physiologic changes to treat sexual dysfunction, OPC-14523 appears to act primarily by increasing sexual desire. Notably, OPC-14523 increased sexual desire in both men and women.

TABLE 9

| CSFQ | Remained | Improved | Worsened | Remained | |
|---|---|---|---|---|---|
| (All Patients - Men | Low | (Low→Normal) | (Normal→Low) | Normal | |
| and Women) | N (%) | N (%) | N (%) | N (%) | p-value |
| Pleasure | | | | | |
| OPC-14523 | 105 (91%) | 6 (5%) | 0 (0%) | 4 (3%) | 0.005 |
| Placebo | 118 (98%) | 0 (0%) | 2 (2%) | 0 (0%) | |
| Desire/Frequency | | | | | |
| OPC-14523 | 86 (75%) | 15 (13%) | 4 (3%) | 10 (9%) | 0.001 |
| Placebo | 106 (88%) | 1 (1%) | 8 (7%) | 5 (4%) | |
| Desire/Interest | | | | | |
| OPC-14523 | 90 (78%) | 8 (7%) | 3 (3%) | 14 (12%) | 0.051 |
| Placebo | 108 (90%) | 5 (4%) | 3 (3%) | 4 (3%) | |
| Arousal/Excitement | | | | | |
| OPC-14523 | 97 (84%) | 7 (6%) | 3 (3%) | 8 (7%) | 0.084 |
| Placebo | 109 (91%) | 2 (2%) | 6 (5%) | 3 (3%) | |
| Orgasm/Completion | | | | | |
| OPC-14523 | 73 (64%) | 14 (12%) | 8 (7%) | 19 (17%) | 0.171 |
| Placebo | 92 (77%) | 12 (10%) | 5 (4%) | 11 (9%) | |
| Total Score | | | | | |
| OPC-14523 | 48 (42%) | 18 (16%) | 3 (3%) | 45 (39%) | 0.010 |
| Placebo | 64 (54%) | 19 (16%) | 10 (8%) | 26 (22%) | |

Shift analysis from baseline to Day 56, LOCF

TABLE 10

| | Remained | Improved | Worsened | Remained | |
|---|---|---|---|---|---|
| CSFQ | Low | (Low→Normal) | (Normal→Low) | Normal | |
| (Men Only) | N (%) | N (%) | N (%) | N (%) | p-value |
| Pleasure | | | | | |
| OPC-14523 | 43 (91%) | 1 (2%) | 0 (0%) | 3 (6%) | 0.103 |
| Placebo | 48 (96%) | 0 (0%) | 2 (4%) | 0 (0%) | |
| Desire/Frequency | | | | | |
| OPC-14523 | 36 (77%) | 6 (13%) | 1 (2%) | 4 (9%) | 0.006 |
| Placebo | 47 (94%) | 0 (0%) | 3 (6%) | 0 (0%) | |
| Desire/Interest | | | | | |
| OPC-14523 | 38 (81%) | 1 (2%) | 0 (0%) | 8 (17%) | 0.063 |
| Placebo | 47 (94%) | 1 (2%) | 1 (2%) | 1 (2%) | |
| Arousal/Excitement | | | | | |
| OPC-14523 | 37 (79%) | 1 (2%) | 2 (4%) | 7 (15%) | 0.105 |
| Placebo | 43 (86%) | 1 (2%) | 5 (10%) | 1 (2%) | |
| Orgasm/Completion | | | | | |
| OPC-14523 | 32 (68%) | 4 (9%) | 4 (9%) | 7 (15%) | 0.123 |
| Placebo | 41 (82%) | 5 (10%) | 3 (6%) | 1 (2%) | |
| Total Score | | | | | |
| OPC-14523 | 19 (40%) | 7 (15%) | 1 (2%) | 20 (43%) | 0.225 |
| Placebo | 21 (42%) | 8 (16%) | 6 (12%) | 15 (30%) | |

Shift analysis from baseline to Day 56, LOCF

TABLE 11

| | Remained | Improved | Worsened | Remained | |
|---|---|---|---|---|---|
| CSFQ | Low | (Low→Normal) | (Normal→Low) | Normal | |
| (Women Only) | N (%) | N (%) | N (%) | N (%) | p-value |
| Pleasure | | | | | |
| OPC-14523 | 62 (91%) | 5 (7%) | 0 (0%) | 1 (1%) | 0.040 |
| Placebo | 70 (100%) | 0 (0%) | 0 (0%) | 0 (0%) | |
| Desire/Frequency | | | | | |
| OPC-14523 | 50 (74%) | 9 (13%) | 3 (4%) | 6 (9%) | 0.052 |
| Placebo | 59 (84%) | 1 (1%) | 5 (7%) | 5 (7%) | |
| Desire/Interest | | | | | |
| OPC-14523 | 52 (76%) | 7 (10%) | 3 (4%) | 6 (9%) | 0.439 |
| Placebo | 61 (87%) | 4 (6%) | 2 (3%) | 3 (4%) | |
| Arousal/Excitement | | | | | |
| OPC-14523 | 60 (88%) | 6 (9%) | 1 (1%) | 1 (1%) | 0.244 |
| Placebo | 66 (94%) | 1 (1%) | 1 (1%) | 2 (3%) | |
| Orgasm/Completion | | | | | |
| OPC-14523 | 41 (61%) | 10 (15%) | 4 (6%) | 12 (18%) | 0.494 |
| Placebo | 51 (73%) | 7 (10%) | 2 (3%) | 10 (14%) | |
| Total Score | | | | | |
| OPC-14523 | 29 (43%) | 11 (16%) | 2 (3%) | 25 (37%) | 0.028 |
| Placebo | 44 (63%) | 11 (16%) | 4 (6%) | 11 (16%) | |

Shift analysis from baseline to Day 56, LOCF

### Example 3

### Comparison of OPC-14523, Bupropion and Escitalopram Effects on Sexual Dysfunction

There is evidence that bupropion reduces the sexual dysfunction in depressed patients, and may be useful as a treatment for sexual dysfunction. Therefore, the data of OPC-14523 effect on sexual dysfunction discussed above were compared to data on the effects of bupropion XL and escitalopram on sexual function reported by Clayton et al. (45^{th} Annual NCDEU Meeting, Boca Raton, Fla. June 6-9, 2005).

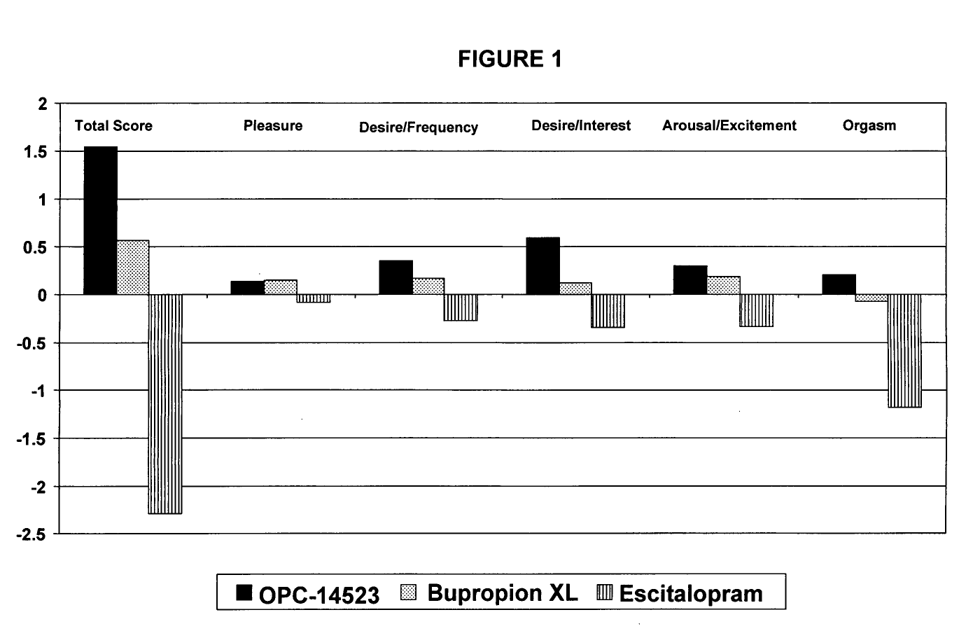

The comparison was based on the change in CSFQ score from baseline to week 8. The changes were assessed by subtracting the placebo score from the treated score at baseline and at week 8, then taking the difference of these two numbers. As shown in FIG. 1, OPC-14523 treatment yielded a larger improvement in sexual function overall, as well as on virtually every CSFQ subscale, compared to bupropion XL treatment. These data suggest the efficacy of OPC-14523 for treating sexual dysfunction is greater than that observed of bupropion XL.

All references cited herein are incorporated by reference. The present invention may be embodied in other specific forms without departing from the spirit or essential attributes thereof and, accordingly, reference should be made to the appended claims, rather than to the foregoing specification, as indication the scope of the invention.

1. A method of treating sexual dysfunction in an individual in need of such treatment, comprising administering to the individual an effective amount of 1-[3-[4-(3-chlorophenyl)-1-piperazinyl]propyl]-5-methoxy-3,4-dihydro-2(1H)-quinolone, a prodrug thereof, or a pharmaceutically acceptable salt thereof.

2. The method according to claim 2, wherein the pharmaceutically acceptable salt is 1-[3-[4-(3-chlorophenyl)-1-piperazinyl]propyl]-5-methoxy-3,4-dihydro-2(1H)-quinolone monomethanesulfonate.

3. The method of claim 1, wherein a measure of the sexual dysfunction includes at least one of reduced sexual interest and reduced sexual desire.

4. The method of claim 3, wherein the sexual dysfunction is hypoactive sexual desire disorder.

5. The method of claim 1, wherein a measure of the sexual dysfunction includes at least one of reduced sexual pleasure and reduced sexual arousal and excitement.

6. The method of claim 5, wherein the sexual dysfunction is male erectile disorder.

7. The method of claim 1, wherein the individual is or has been diagnosed with one of major depressive disorder and generalized anxiety disorder.

8. The method of claim 1, wherein 1-[3-[4-(3-chlorophenyl)-1-piperazinyl]propyl]-5-methoxy-3,4-dihydro-2(1H)-quinolone, or a pharmaceutically acceptable salt thereof, is administered orally.

9. The method of claim 1, wherein the individual is a human being.
